(19) United States
(12) Patent Application Publication
(10) Pub. No.: US 2019/0276878 A1
(43) Pub. Date: Sep. 12, 2019

(54) SINGLE LABELED FLUORESCENT SMART PROBE FOR IN VITRO DETECTION

(71) Applicant: King Fahd University of Petroleum and Minerals, Dhahran (SA)

(72) Inventor: Sulayman Oladepo, Dhahran (SA)

(73) Assignee: King Fahd University of Petroleum and Minerals, Dhahran (SA)

(21) Appl. No.: 16/210,411

(22) Filed: Dec. 5, 2018

Related U.S. Application Data
(60) Provisional application No. 62/641,081, filed on Mar. 9, 2018.

(51) Int. Cl. C12Q 1/6816; C12Q 1/6886

(52) U.S. Cl. CPC C12Q 1/6816; C12Q 2600/158; C12Q 2600/178; C12Q 1/6886

(57) ABSTRACT

A smart nucleic acid probe for the detection and quantification miRNA is disclosed. Also, a method of early detection and diagnosing cancer is described. The method is highly specific, sufficiently sensitive, and simple to carry out.

### CROSS-REFERENCE TO RELATED APPLICATION

This application claims priority benefit from provisional application 62/641,081 filed Mar. 9, 2018 incorporated herein by reference in its entirety.

### STATEMENT OF FUNDING ACKNOWLEDGEMENT

This project was funded by the Deanship of Scientific Research (DSR) at King Fand University of Petroleum and Minerals (KFUPM) through DSR startup research grant SR151001.

### SEQUENCE LISTING

The instant application contains a Sequence Listing which has been filed electronically in ASCII format and is hereby incorporated by reference in its entirety. Said ASCII copy, created on Jul. 17, 2018, is named 512187US_SL.txt and is 1,127 bytes in size.

### STATEMENT REGARDING PRIOR DISCLOSURE BY THE INVENTORS

Aspects of this technology are described in an article “Design and characterization of singly labeled fluorescent smart probe for in vitro detection of miR-21” Applied Spectroscopy (2018) Vol. 72 (1) 79-88. The article was published online Oct. 25, 2017,

### BACKGROUND OF THE INVENTION

### Field of the Disclosure

The present invention is related to a nucleic acid smart probe (SP) for detecting a specific target DNA or RNA, in particular, micro-RNAs such as human miRNA-21of SEQ ID NO: 1 and its variants. The nucleic acid smart probe is used as a tool for detecting miRNA for the early detection of neurological diseases, viral infection, and cancers.

### Description of Related Art

Micro-RNAs (miRNAs) are naturally occurring, small, non-coding RNA molecules that regulate several biological processes [Lagos-Quintana et al. Science. 2001. 294(5543): 853-858; and Zhu et al. J. Biol. Chem. 2007. 282(19): 14328-14336]. They play an important role in gene expression by targeting messenger RNAs (mRNAs) in a sequence-specific manner [Lagos-Quintana et al.; Fabani et al. RNA. 2008. 14(2): 336-346; Pillai et al. RNA. 2005. 11(12): 1753-1761; and Zamore et al. Science. 2005. 309(5740): 1519-1524]. Also, they play a significant role in the initiation and progression of several diseased states, including neurological diseases, viral infections, and cancer [Zhu et al.; Cissell et al. Anal. Chem. 2008. 80(7): 2319-2325; and Croce et al. Nature Rev. Genet. 2009. 10(10): 704-714]. For instance, several miRNAs have been implicated in cancer progression and prognosis (Croce et al.). Consequently, miRNAs are considered important diagnostic and prognostic bio-markers [Cissell et al., and Catuogno et al. Cancers. 2011. 3(2): 1877-1898] and they can also be harnessed for therapeutic purposes [Cissell et al. and Croce et al]. In the case of cancer, miRNAs may act either as tumor suppressors or as oncogenes [Zhu et al. and Chen et al. and N. Engl. J. Med. 2005. 353: 1768-1771]. MicroRNA-21 (miR-21) of SEQ ID NO: 1 has been specifically implicated in several types of cancer, including breast, lung, prostate, head and neck, stomach, ovarian, and cervical cancers, as well as leukemia [Croce et al. and Catuogno et al.]. Given the diagnostic and therapeutic significance of miRNAs, several analytical detection platforms have been developed for sensitive determination of miRNAs in vitro and in vivo.

The traditional methods used for detection of miRNAs are based on polymerase chain reaction (PCR), northern blotting, in situ hybridization, and microarray techniques [Cissell et al.; Catuogno et al.; Jiang et al. Anal. Chim. Acta. (2016). 943: 114-122; and Liu et al. Anal. Chem. 2012. 84(12): 5165-5169]. An amplification method of quantitative reverse-transcriptase polymerase chain reaction (qRT-PCR) has also been used for miRNA detection [Chen et al. Nucleic Acids Res. 2005, 33(20): e179; and Zhang et al. Theranostics. 2015. 5(7): 733-745]. While PCR-based methods are highly selective and specific, they are time-consuming and labor-intensive, and success relies on isolation of purified total RNA [Zhang et a. Anal. Chem. 2016. 88(2): 1294-1302; and Wang et al. Anal. Chem. 2012. 84(16): 7037-7042]. Although it is still being used as a standard method of analysis, northern blotting has low sensitivity, is semi-quantitative, cumbersome, and requires a large amount of sample [Cissell et al.; Catuogno et al.; and Liu et al.]. Despite the high throughput of microarray methods, they have limited sensitivity, selectivity, and specificity. [Cissell et al.; Catuogno et al.; and Wang et al.]. An electrochemical technique involving the use of a pencil graphite electrode has been shown to give picomolar detection limit for miR-21 of SEQ ID NO: 1 [Kilic et al. Biosens. Bioelectron. 2012. 38(1): 195-201]. However, this technique has several washing steps and the overall protocol is complicated. A colorimetric detection method based on the use of gold nanoplasmonic particles has also been used for the detection of miR-21 of SEQ ID NO: 1 [Park et al. Chem. Comm. 2014. 50(11): 1366-1368]. Changes in scattering color that are induced by miR-21 of SEQ ID NO: 1 when hybridized to its complementary target that is tethered onto gold nanoparticles is used as the basis for detection. This approach works well and provides low detection limits, but the requirement for fabrication of nanoplasmonic particles and the assembly of the particles conjugated with oligonucleotide probes as a condition for detection may be unattractive for easy and simple detection. Furthermore, a surface-enhanced Raman spectroscopy (SERS)-based assay method has been demonstrated for the detection of miR-21 of SEQ ID NO: 1 [Guven et al. Analyst. 2014. 139(5): 1141-1147]. The technique was reported to give detection limit in the subnanomolar range, but the hybridization/detection procedure is complicated. The use of a bioluminescent enzyme-based hybridization assay method for sensitive miR-21 of SEQ ID NO: 1 detection has also been reported (Cissell et al.). This method represents an excellent approach for miRNA detection, and although several labeling steps are involved, it provides a very low detection limit and is capable of direct determination of miR-21 of SEQ ID NO: 1 in cell extracts.

New generation nucleic acid amplification methods present unique advantages for miRNA detection [Du et al. Chem. Comm. 2016. 52(86): 12721-12724]. Amplification methods including enzymatic repairing amplification [Zhou et al. Anal. Chem. 2014, 86(14): 6763-6767], strand displacement amplification (Zhang 2016), isothermal amplification methods [Liu et al.; Duan et al. J. Am. Chem. Soc. 2013. 135(12): 4604-4607; and Cheng et al. Biosens. Bioelectron. 2016. 85: 891-896], circular exponential amplification (Wang et al.), and rolling circle amplification methods have been proposed for miRNA detection (Jiang et al. 2016). In addition, a ligation based, loop-mediated amplification (ligation-LAMP) strategy has been reported (Du et al.). All methods have enhanced sensitivity for miRNA detection, and despite the complexity of their reaction or hybridization mixtures, they provide excellent specificity for their target miRNAs. Also, the methods show potential for in vivo determination of miRNAs. Typically, fluorescence-based methods provide high sensitivity [El-Yazbi et al. Anal. Chem. 2013. 85(9): 4321-4327] and new generation amplification methods couple their respective amplification strategies with fluorescence detection. The aforementioned limitations of northern blotting and microarray methods combined with the complex nature of colorimetric, nanoplasmonic, and nucleic acid amplification methods highlight a need for simple, fast, sensitive, and specific analytical detection method for miRNA.

Homogeneous assay methods that are based on smart probes (SPs) may be adopted for miRNA detection. SP-based homogeneous assay methods are specific, simple, and fast, and have high sensitivity. Smart probes are nucleic acids having hairpin-shaped structure comprising inherent signaling property due to their hairpin conformation [Knemeyer et al. Anal. Chem. 2000. 72(16): 3717-3724; Stohr et al. Anal. Chem. 2005. 77(22): 7195-7203; Marme et al. Anal. Bioanal. Chem. 2007. 388(5-6): 1075-1085; Misra et al. Anal. Biochem. 2007. 364(1): 86-88; Friedrich et al. Proc. SPIE. 2007, 6444: 64440M; and Heinlein et al. J. Phys. Chem. B. 2003. 107(31): 7957-7964]. They consist of a stem-loop structure, with a fluorophore on one end of the nucleic acid sequence and a quencher on the other. The structure of the SP is analogous to molecular beacon hairpins (MBs) [Tyagi et al. Nat. Biotechnol. 1996. 14(3): 303-308; and Bonnet et al. Proc. Natl. Acad. Sci. USA. 1999. 96(11): 6171-6176], but the quencher in MBs is replaced with multiple guanine residues [Knemeyer et al. Anal. Chem. 2000. 72(16): 3717-3724; Stohr et al. Anal. Chem. 2005. 77(22): 7195-7203; Marme et al. Anal. Bioanal. Chem. 2007. 388(5-6): 1075-1085; Misra et al. Anal. Biochem. 2007. 364(1): 86-88; Friedrich et al. Proc. SPIE. 2007. 6444: 64440M; and Heinlein et al. J. Phys. Chem. B. 2003. 107(31): 7957-7964, incorporated herein by reference]. Thus, in contrast to MBs, SPs only have one extrinsic label (the fluorophore) on one end of the stem, while quenching is achieved using intramolecular guanine residues on the other end of the stem hybrid [Knemeyer et al. and Stohr et al.]. One major advantage of SP over MB is that probes that are inadvertently left unlabeled with extrinsic quencher (as is possible in the case of MB) do not contribute to the measured signal [Knemeyer et al.]. In the hairpin form, the fluorophore and the quenching guanine residues are in close proximity and the fluorescence is quenched due to energy transfer from the fluorophore to the quencher guanidine residues [Stohr et al.; Steenken et al. J. Am. Chem. Soc. 1997. 119(3): 617-618; and Seidel et al. J. Phys. Chem. 1996. 100(13): 5541-5553, incorporated herein by reference]. When the SP recognizes and hybridizes to a complementary target sequence, the probe is no longer a hairpin structure, which spatially separates the fluorophore and the quencher, and thereby switching on the fluorescence. Due to their excellent signaling property, SP has been successfully used for various detection assays, including detection of ultraviolet (UV)-induced DNA damage [Oladepo et al. Anal. Bioanal. Chem. 2010. 397(7): 2949-2957; and Nair et al. Photochem. Photobiol. 2013. 89(4): 884-890, incorporated herein by reference], real-time monitoring of the activity and kinetics of T4 polynucleotide kinase [Song et al. Anal. Chem. 2009. 81(4): 1383-1388, incorporated herein by reference], detection of ATP [Ma et al. Anal. Biochem. 2012. 429(1): 8-10, incorporated herein by reference], monitoring of DNA methyltransferase activity [Jin et al. Mol. Cell. Probes. 2016. 30(3): 185-187, incorporated herein by reference], and monitoring of DNA polymerase fidelity [Song et al. Biosens. Bioelectron. 2011. 26(5): 2699-2702]. SP's have been shown to have high sensitivity and are capable of recognizing a single-base mismatches (Oladepo et al.).

Accordingly, it is an object of the present disclosure to provide a nucleic acid smart probe for the detection of miRNA, in particular, miR-21 of SEQ ID NO: 1 and variants thereof, a 22 nucleotide miRNA, which is known to be upregulated in several types of human cancer including, but not limited to leukemia, breast, colon, pancreatic, lung, prostate, liver, and stomach cancers (Croce et al. and Catuogo et al.). It is a further objective of this disclosure to provide a homogeneous assay method utilizing the SP's as diagnostic tools for early detection of cancer.

### SUMMARY

A first aspect of the invention is directed to a nucleic acid smart probe comprising:
(a) a complementary nucleic acid sequence to a target sequence of interest,
(b) a nucleic acid sequence A of 5 to 15 nucleotides bonded to the 3′-end of the complementary sequence of (a),
(c) a nucleic acid sequence B of 5 to 15 nucleotides bonded at the 5′-end of the complementary sequence of (a),
(d) a GGG sequence bonded at the 5′- or the 3′-end of the nucleic acid sequence A or B, and
(e) a fluorescence moiety bonded at the 5′- or the 3′-end of the nucleic acid sequence A or B,

wherein the nucleic acid sequences A and B are complementary sequences and form a stem of a hairpin structure.

In a preferred embodiment of the nucleic acid probe, the fluorescence moiety is at the 5′-end and the GGG sequence at the 3′-end.

In another preferred embodiment, the fluorescence moiety is at the 3′-end and the GGG sequence at the 5′-end.

In another preferred embodiment, the melting temperature (Tₘ) of the probe is in the range 57° C. to 67° C.

In a more preferred embodiment, the Tₘ of the probe is about 63° C.

In another preferred embodiment, the melting temperature Tₘ of the probe-target nucleic acid duplex is in the range 43° C. to 50° C.

In another preferred embodiment the fluorescence moiety is 6-carboxyflurescein.

In a more preferred embodiment, the Tₘ of the probe -target nucleic acid is about 47° C.

In a more preferred embodiment, the target nucleic acid of interest is human miR-21 of SEQ ID NO: 1.

In even more preferred embodiment the probe, the nucleic acid sequence of the probe has the nucleic acid sequence of SEQ ID NO: 2.

In the most preferred embodiment, the nucleic acid sequence of the probe is SEQ ID NO: 2, the fluorescence moiety is 6-carboxyflurescein at the 5′-end, and the GGG sequence is at the 3′-end.

A second aspect of the invention is directed to detecting and/or quantifying a target nucleic acid sequence in a biological sample comprising:

(a) contacting the smart probe of invention with a biological sample, and

(b) measuring the increase in fluorescence intensity.

In a preferred embodiment, the target nucleic acid is miRNA.

In a more preferred embodiment, the target nucleic acid sequence is miR-21 of SEQ ID NO: 1.

In another preferred embodiment, the fluorescence is measured at a temperature between the Tₘ of the probe and Tₘ of the probe-target nucleic acid duplex.

In a more preferred embodiment, the fluorescence is measured at a temperature in the range 45° C. and 56° C.

In the most preferred embodiment, the fluorescence is measured at 52° C.

A third aspect of the invention is directed a method of diagnosing or detecting cancer in a subject comprising:
(a) contacting a biological sample of the subject with the probe of claim 9,
(b) measuring the increase in the fluorescence of the probe,
(c) determining the concentration of miR-21 of SEQ ID NO: 1 in the biological sample, and
(d) diagnosing the presence of cancer when the concentration of miR-21 of SEQ ID NO: 1 in the subject is greater than the range of concentrations of miR-21 of SEQ ID NO: 1 determined in cancer free population.

In a preferred embodiment of the method, the cancer is selected from the group consisting of leukemia, breast, colon, pancreatic, lung, prostate, liver, and stomach cancers.

In a more preferred embodiment, the method further comprises quantifying the amount of miRNA-21 of SEQ ID NO: 1 in the biological sample.

In another preferred embodiment, the fluorescence is measured at a temperature in the range 45° C. and 56° C.

In a more preferred embodiment, the fluorescence is measured at a temperature of 52° C.

In another preferred embodiment, the subject is a mammal.

In a more preferred embodiment, the mammal is human.rs.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows the nucleotide sequence of human miR-21 target sequence of SEQ ID NO: 1 and the smart probes of SEQ ID NO: 2 as well as the hairpin structure of the smart probe. The stem sequences of the smart probe are underlined in the SP-RNA1 duplex (bottom right), which form the stem of the hairpin.

FIG. 2A shows temperature-dependent fluorescence spectra of the SP of SEQ ID NO: 2.

FIG. 2B: shows temperature-dependent fluorescence spectra of the hybrid of SP of SEQ ID NO: 2 and RNA1 of SEQ ID NO: 1.

FIG. 2C: shows temperature-dependent fluorescence spectra of the hybrid of SP of SEQ ID NO: 2 and RNA2 of SEQ ID NO: 3.

FIG. 2D: shows temperature-dependent fluorescence spectra of the hybrid of SP of SEQ ID NO: 2 and RNA3 of SEQ ID NO: 4.

FIG. 2E shows the thermal transition profiles of SP of SEQ ID NO: 2 (squares), SP of SEQ ID NO: 2-RNA1 of SEQ ID NO: 1 duplex (circles), SP of SEQ ID NO: 2-RNA2 of SEQ ID NO: 3 duplex (diamonds), and SP of SEQ ID NO: 2-RNA3 of SEQ ID NO: 4 duplex (hexagons) duplexes.

FIG. 3A shows fluorescence spectra of SP of SEQ ID NO: 2, SP of SEQ ID NO: 2 duplex with miR-21 of SEQ ID NO: 1 (RNA1), SEQ ID NO: 3 (RNA2), and SEQ ID NO: 4 (RNA3) measured at 52° C.

FIG. 3B shows the fluorescence intensity of SP of SEQ ID NO: 2, SP of SEQ ID NO: 2 duplex with miR-21 of SEQ ID NO: 1 (RNA1), SEQ ID NO: 3 (RNA2), and SEQ ID NO: 4 (RNA3) measured at 52° C.

FIG. 4A shows the time-dependent fluorescence intensity of a solution of SP of SEQ ID NO: 2 duplex with miR-21 of SEQ ID NO: 1 (SP-RNA1) measured at 37° C.

FIG. 4B shows the time-dependent fluorescence intensity of a solution of SP of SEQ ID NO: 2 duplex with miR-2# of SEQ ID NO: 1 (SP-RNA1) measured at 20° C.

FIG. 5A shows fluorescence intensities of 500 nM SP of SEQ ID NO: 2 at varying concentrations of RNA1 of SEQ ID NO: 1. Curve becomes non-linear beyond 500 nM RNA1 target concentration. The linear portion of the curve is shown in FIG. 5B.

FIG. 5B shows fluorescence intensity of 500 nM of SP of SEQ ID NO: 2 at varying concentration of RNA1 of SEQ ID NO; 1 in the range 0-500 nm.

FIG. 6A shows fluorescence spectra of incubated duplexes of SP of SEQ ID NO: 2 and RNA1 of SEQ ID NO: 1, RNA2 of SEQ ID NO: 3, and RNA3 of SEQ ID NO: 4 were obtained at 20° C.

FIG. 6B shows fluorescence intensities at 520 nm of each duplex at 20° C. obtained from the spectra shown in FIG. 6A. Intensity of SP-RNA2 is about 90% that of SP-RNA1 while that of SP-RNA3 is about 85% that of SP-RNA1. Error bars represent standard deviation of three replicate measurements.

FIG. 7A shows fluorescence spectra of duplexes of SP of SEQ ID NO: 2 and RNA1 of SEQ ID NO: 1, RNA2 of SEQ ID NO: 3, and RNA3 of SEQ ID NO: 4 were obtained at 52° C. after incubation at 37° C.

FIG. 7B shows fluorescence intensities at 520 nm of each duplex at 52° C. obtained from the spectra shown in FIG. 7A. Intensity of SP duplex of SEQ ID NO: 2 and RNA1 of SEQ ID NO: 1 is about 230% of those of RNA2 SEQ ID NO: 3 and RNA3 of SEQ ID NO: 4. Error bars represent standard deviation of three replicate measurements.

FIG. 8A shows fluorescence spectra of duplexes of SP of SEQ ID NO: 2 and RNA1 of SEQ ID NO: 1, RNA2 of SEQ ID NO: 3, and RNA3 of SEQ ID NO: 4 were obtained at 37° C.

FIG. 8B shows fluorescence intensities at 520 nm of each duplex at 37° C. obtained from the spectra shown in FIG. 8A. Intensity of the duplex of SP of SEQ ID NO: 2 and RNA1 of SEQ ID NO: 1 is about 160% of that of RNA2 of SEQ ID NO: 3 and about 150% that of SP-RNA3 of SEQ ID NO: 4. Error bars represent standard deviation of three replicate measurements.

### DETAILED DESCRIPTION

Embodiments of the present disclosure will now be described more fully hereinafter with reference to the accompanying drawings, in which some, but not all embodiments of the disclosure are shown. The present disclosure will be better understood with reference to the following definitions.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Also, the use of “or” means “and/or” unless stated otherwise. Similarly, “comprise,” “comprises,” “comprising” “include,” “includes,” and “including” are interchangeable and not intended to be limiting.

As used herein, the term “compound” is intended to refer to a chemical entity, whether in a solid, liquid or gaseous phase, and whether in a crude mixture or purified and isolated.

As used herein, the term “salt” refers to derivatives of the disclosed compounds, monomers or polymers wherein the parent compound is modified by making acid or base salts thereof. Exemplary salts include, but are not limited to, mineral or organic acid salts of basic groups such as amines, and alkali or organic salts of acidic groups such as carboxylic acids. The salts of the present disclosure can be synthesized from the parent compound that contains a basic or acidic moiety by conventional chemical methods. Generally such salts can be prepared by reacting the free acid or base forms of these compounds with a stoichiometric amount of the appropriate base or acid in water or in an organic solvent, or in a mixture of the two; generally non-aqueous media like ether, ethyl acetate, ethanol, isopropanol, or acetonitrile are preferred.

As used herein, the term “about” refers to an approximate number within 20% of a stated value, preferably within 15% of a stated value, more preferably within 10% of a stated value, and most preferably within 5% of a stated value. For example, if a stated value is about 8.0, the value may vary in the range of 8±1.6, ±1.0, ±0.8, ±0.5, ±0.4, ±0.3, ±0.2, or ±0.1.

As used herein, “% identity” or “sequence identity” of two nucleic acid sequences, is determined using the algorithm of Karlin and Altschul [Proc. Natl. Acad. Sci. USA, 87:2264 2268, 1990)], modified as in Karlin and Altschul [Proc. Natl. Acad. Sci. USA, 90:5873 5877, 1993]. Such an algorithm is incorporated into the NBLAST and XBLAST programs of Altschul et al. [J. Mol. Biol., 215:403 410, 1990]. BLAST nucleotide searches are performed with the NBLAST program, score=100, wordlength=12. BLAST protein searches are performed with the) XBLAST program, score=50, wordlength=3. To obtain gapped alignment for comparison purposes GappedBLAST is utilized as described in Altschul et al. [Nucleic Acids Res., 25:3389 3402, 1997]. When utilizing BLAST and GappedBLAST programs the default parameters of the respective programs (e.g., XBLAST and NBLAST) are used to obtain nucleotide sequences homologous to a desired nucleic acid molecule.

As used herein, the term “specifically hybridizable” and “complementary” are used to indicate a sufficient degree of complementarity such that stable and specific binding occurs between a smart probe of the invention and a target DNA or RNA molecule. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

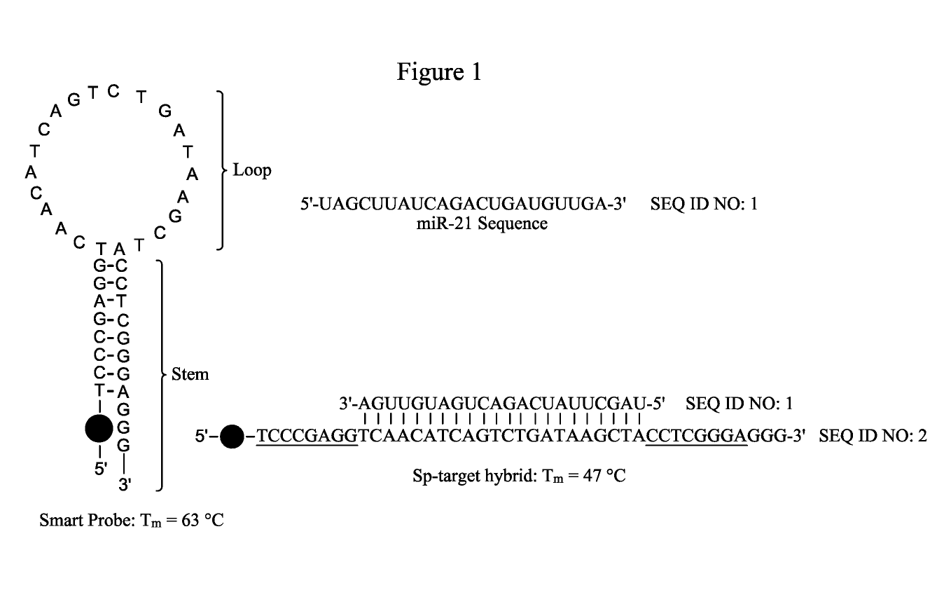

As used herein the term “smart probe” is a nucleic acid probe having the ability to form a hairpin structure comprising a loop region, stem region, a fluorophore and a GGG sequence (G-triplet). See FIG. 1. The loop region comprises a nucleic acid sequence which is complementary to a target sequence.

As used herein the term “target” or “target sequence” refers to a nucleic acid sequence which is complementary to the loop sequence of the smart probe.

A first aspect of the invention is directed to a nucleic acid smart probe comprising:
(a) a complementary nucleic acid sequence to a target sequence of interest,
(b) a nucleic acid sequence A of 5 to 15 nucleotides bonded to the 3′-end of the complementary sequence of (a),
(c) a nucleic acid sequence B of 5 to 15 nucleotides bonded at the 5′-end of the complementary sequence of (a),
(d) a GGG sequence bonded at the 5′- or the 3′-end of the nucleic acid sequence A or B, and
(e) a fluorescence moiety bonded at the 5′- or the 3′-end of the nucleic acid sequence A or B,

wherein the nucleic acid sequences A and B are complementary sequences and form a stem of a hairpin structure.

In a preferred embodiment of the nucleic acid probe, the fluorescence moiety is at the 5′-end and the GGG sequence at the 3′-end.

The smart probe is capable of forming a hairpin structure and comprises a recognition sequence for a target nucleic acid, and two complementary sequences which are attached to the 5′-end and the 3′-end forming the hairpin stem (see FIG. 1). In addition, the smart probe comprises a fluorescence probe and a quenching G-triplet at the 5′- and the 3′-ends, respectively. The smart probe is designed to identify and quantify any target DNA or RNA sequence. The DNA or RNA sequence may contain any number of nucleotides, which may be a non-coding sequence or a coding sequence of any peptide, enzyme or protein, and comprises a target sequence. The target sequence may have any number of nucleotides, preferably in the range of 10 to 100 nucleotides in length, more preferably in the range of 15 to 50 nucleotides, and most preferably in the range of 20 to 30 nucleotides in length. In a particularly, preferred embodiment, the target sequence is about 22 nucleotide in length.

In a preferred embodiment, the probe of the invention is particularly designed to identify and quantify microRNA (miRNA). As used herein, miRNA is a small non-coding RNA molecule containing about 22 nucleotides found in plants and animals as well as in some viruses. The function of miRNA is to silence mRNA as a post translation regulator. While most of miRNA's are located inside cells, some miRNA are found in biological fluids and are known as circulating miRNA or extracellular miRNA. They are encoded by nuclear DNA of plants, animal and viruses, and function by base-pairing with mRNA complementary sequence. The base pairing of miRNA to a target mRNA leads to disabling the mRNA by cleavage of the mRNA, destabilizing the mRNA by shortening the polyA tail or causing less efficient translation of the mRNA into protein by the ribosome. The human genome encodes over 10,000 miRNA which are abundant in many mammalian cell types and appear to target about 60% of the genes of humans and other mammals. The miRNA's are named by convention with the prefix “miR” followed by a dash and a number. miRNA's with nearly identical sequences except for one or two nucleotides are annotated with an additional lower case letter. For example, miR-124a is closely related to miR-124b. Also, the name miRNA may be preceded by three letters indicating the species from which the miRNA is obtained. For example, has-miR-124 refers to Homo sapiens miR-124.

Not only are miRNA involved in the normal functioning of eukaryotic cells, but they are also associated with many diseases including but not limited to hereditary diseases, cancers, cardiovascular diseases, kidney diseases, alcoholism, obesity, viral infection, and nervous system diseases. Many of the diseases are associated with miRNA abnormalities including mutation in the miRNA sequence or its precursor, and higher or lower levels of a particular miRNA in biological samples. For example, a mutation in the precursor of miR-96 causes hereditary progressive hearing loss, and deletion of the miR-17 cluster causes skeletal and growth defects. The first human disease known to be associated with miRNA deregulation was chronic lymphocytic leukemia. Also, many other miRNAs have links with cancer and accordingly are sometimes referred to as “Oncomir”. In malignant B cells, miRNAs participate in pathways fundamental to B cell development like B-cell receptor (BCR) signaling, B-cell migration/adhesion, cell-cell interactions in immune niches and the production and class-switching of immunoglobulins. miRNAs influence B cell maturation, generation of pre-marginal zone, follicular, B1, plasma and memory B cells. Hepatocellular carcinoma cell proliferation may arise from miR-21 of SEQ ID NO: 1 interaction with MAP2K3, a tumor repressor gene. Optimal treatment for cancer involves accurately identifying patients for risk-stratified therapy. Those with a rapid response to initial treatment may benefit from truncated treatment regimens, showing the value of accurate disease response measures. Cell-free miRNA are highly stable in blood, overexpressed in cancer, and quantifiable within a diagnostic laboratory. In classical Hodgkin lymphoma, plasma miR-21 of SEQ ID NO: 1, miR-494, and miR-1973 are promising disease response biomarkers. Circulating miRNAs have the potential to assist clinical decision making and aid interpretation of positron emission tomography combined with computerized tomography. They can be performed at each consultation to assess disease response and detect relapse. Thus, it is one of the objects of the invention to provide a smart probe for the detection, quantification and identification of abnormalities of miRNA.

Encoded by the MIR21lgene, miR-21 was one of the first mammalian miRNAs identified. The mature miR-21 sequence is strongly conserved throughout evolution. The human miR-21 of SEQ ID NO: 1 gene is located on the plus strand of chromosome 17q23.2 (55273409-55273480) within a coding gene TMEM49 encoding vacuole membrane protein. Despite being located in intronic regions of a coding gene in the direction of transcription, it has its own promoter regions and forms a ˜3433-nt long primary transcript of miR-21 (known as pri-miR-21) which is independently transcribed. The stem-loop precursor of miR-21(pre-miR-21) resides between nucleotides 2445 and 2516 of pri-miR-21. Pri-miR-21 is cut by the endonuclease Drosha in the nucleus to produce pre-miR-21, which is exported into the cytosol. This pre-miR-21 is then cut into a short RNA duplex by Dicer in the cytosol. Although abundance of both strands is equal by transcription, only one strand is selected for processing to mature miR-21 of SEQ ID NO: 1 based on the thermodynamic stability of each end of the duplex. The other strand, designated miR-21*, is generally degraded. Mature miRNA is then loaded into miRNA ribonucleoprotein complex RISC (RNA-induced silencing complex) and guided to target mRNAs with near perfect complimentarily at 3′UTR.

A number of targets for miR-21 of SEQ ID NO: 1 have been identified and most of them are tumor suppressor genes including, but not limited to, ANP32A, BTG2, Bc12, P12/CDK2AP1, HNRPK<IL-12p35, JAG1, MEF2C, hMSH2, and PDCD4. miR-21 of SEQ ID NO: 1 is one of the most frequently upregulated miRNAs in solid tumors, and its high levels were first described in B cell lymphomas. Overall, miR-21 of SEQ ID NO: 1 is considered to be a typical “onco-miR”, which acts by inhibiting the expression of phosphatases, which limit the activity of signaling pathways such as AKT and MAPK. miR-21 of SEQ ID NO: 1 can be transcriptionally activated by NF-κB and downregulate phosphatases PDCD4 and PTEN. A recent study also suggested that miR-21 of SEQ ID NO: 1 could provide a link between inflammation and cancer. As most of the targets of miR-21 of SEQ ID NO: 1 are tumor suppressors, miR-21 of SEQ ID NO: 1 and variants thereof are associated with a wide variety of cancers including that of breast cancer, ovarian, cervix, colon, lung, liver, brain, esophagus, prostate, and thyroid. A 2014 meta-analysis of 36 studies identified circulating miR-21 of SEQ ID NO: 1 as a biomarker of various human carcinomas and a potential tool for early diagnosis. miR-21 of SEQ ID NO: 1 expression was associated with survival in 53 triple negative breast cancer patients.

Also, miR-21 of SEQ ID NO: 1 and variants thereof have been shown to play an important role in the development of heart disease. It is one of the miRNAs whose expression is increased in failing murine and human hearts. Further, inhibition of miRNAs in mice using chemically modified and cholesterol-conjugated miRNA inhibitors was shown to inhibit interstitial fibrosis and improve cardiac function in a pressure overload cardiac disease mice model. Surprisingly, miR-21 global knock-out mice did not show any overt phenotype when compared with wild type mice with respect to cardiac stress response. Similarly, short (8-nt) oligonucleotides designed to inhibit miR-21 of SEQ ID NO: 1 or variant thereof could not inhibit cardiac hypertrophy or fibrosis. In another study with a mouse model of acute myocardial infarction, miR-21 expression was found to be significantly lower in infarcted areas and overexpression of miR-21 in those mice via adenovirus-mediated gene transfer decreased myocardial infarct size.

A preferred target nucleic acid sequence is a nucleic acid sequence having sequence identity in the range of 50%, more preferably having sequence identity in the range of 75% to 100%, even more preferably, in the range of 85 to 100%, and most preferably 90% to 100% of SEQ ID NO: 1.

The smart probe is designed in such a way to form a hairpin structure comprising a sequence recognition loop, a double helical stem, G-triplet, and a fluorophore. The formation of the hairpin structure brings the fluorophore from one end of the probe at the 5′- or 3′-end to spatially close proximity to the quencher G-triplet at the other end of the probe. The fluorophore is any fluorescent moiety which can be covalently attached to either the 5′-end or the 3′-end by well-known methods in the art and is quenched by the G-triplet. Examples of fluorophores include but are not limited to cyanine dyes such as Cyt, Cy3, Cy3B, Cy3.5, Cy5, Cy5.5, Cy7; JOE, TET, HEX, TRITC, RTM., xanthine derivatives including Fluorescein and derivatives thereof such as 6-FAM (6-carboxyfluorescein), Rhodamine and derivatives thereof such as X-Rhodamine, Rhodamine 6G, and Lissamine Rhodamine B, orange green, eosin, and Texas red; dansyl derivatives or any other naphthalene derivatives, coumarin and derivatives thereof such as methoxycoumarin, aminocoumarin, and hydroxycoumarin; Lucifer Yellow; Pacific Blue; Pacific Orange; Cascade Blue; FluorX; TruRed; PerCP; and Red 613.

In a preferred embodiment, the fluorophore is xanthine derivative such as but not limited to Fluorescein, Rhodamine and derivatives thereof such as X-Rhodamine, Rhodamine 6G, and Lissamine Rhodamine, orange green, eosin, and Texas and more preferably 6-carboxyfluorescein.

The fluorophore may be attached to the 3- or the 5′-end. Similarly, the quenching sequence GGG (G-triplet) may be located at either end. The only requirement is that the fluorophore and the G-triplet are located at the opposite ends of the smart probe.

The two complementary sequences located at the opposite end of the smart probe (see FIG. 1) may have any sequence as long as the probe is capable of assuming the hairpin structure at biological and/or ambient temperature. For some application, it may be desirable to have the hairpin structure denature at a melting temperature (Tₘ) higher than that of the duplex formed between the target DNA or RNA and the smart probe. In a preferred embodiment, the difference in temperature between the Tₘ of the smart probe and the duplex formed by the target nucleic acid is in the range of 5° C. and 25° C., more preferably in the range 10° C. and 20° C., and most preferably 13° C. and 18° C. The melting temperature of the probe is dependent on the base-pair composition of a duplex and the length of the duplex. The melting temperature increases with increasing the number GC pairs content in a duplex and increasing the length of the duplex. The number of base pairs in the stem of the hairpin structure may be in the range 4 to 20 base pairs, preferably in the range of 6 to 15 base pairs, more preferably in the range of 7-13 base pairs, and most preferably in the range of 8 to 12 base pairs. In a preferred embodiment, the melting temperature (Tₘ) of the probe is in the range 55° C. to 70° C., more preferably 57° C. to 67. ° C., and most preferably in the range of 60° C. to 65° C.

In a preferred embodiment, the Tₘ of the probe-target nucleic acid duplex is in the range of 35° C. to 55° C., preferably in the range 38° C. to 52° C., more preferably in the range 42° C. to 49° C., and most preferably about 47° C.

In a preferred embodiment, the smart probe comprises a nucleic acid sequence having at least 50%, preferably at least 60%, more preferably at least 70%, even more preferably at least 80%, and most preferably 90% sequence identity to SEQ ID NO: 2. In a particularly preferred embodiment, the smart probe has the nucleic acid sequence of SEQ ID NO: 2.

A second aspect of the invention is directed to an analytical method for detecting and quantifying nucleic acid marker in a biological sample. The method comprises contacting the biological sample with the smart probe disclosed herein under conditions which allow the specific hybridization of the probe to the target nucleic acid sequence, and measuring the fluorescence produced by the hybrid nucleic acid.

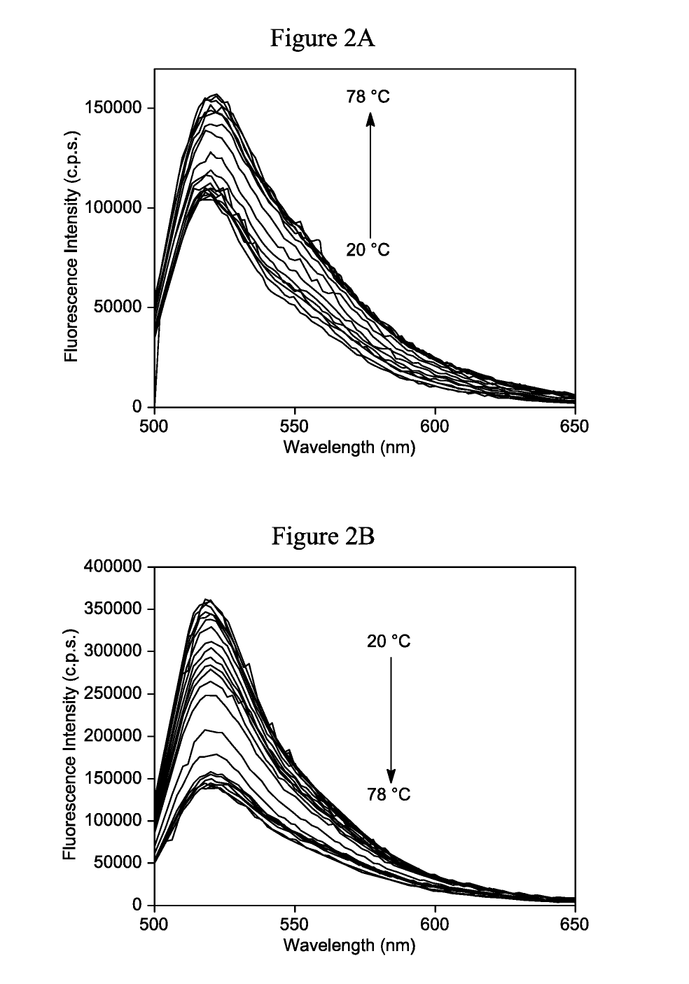

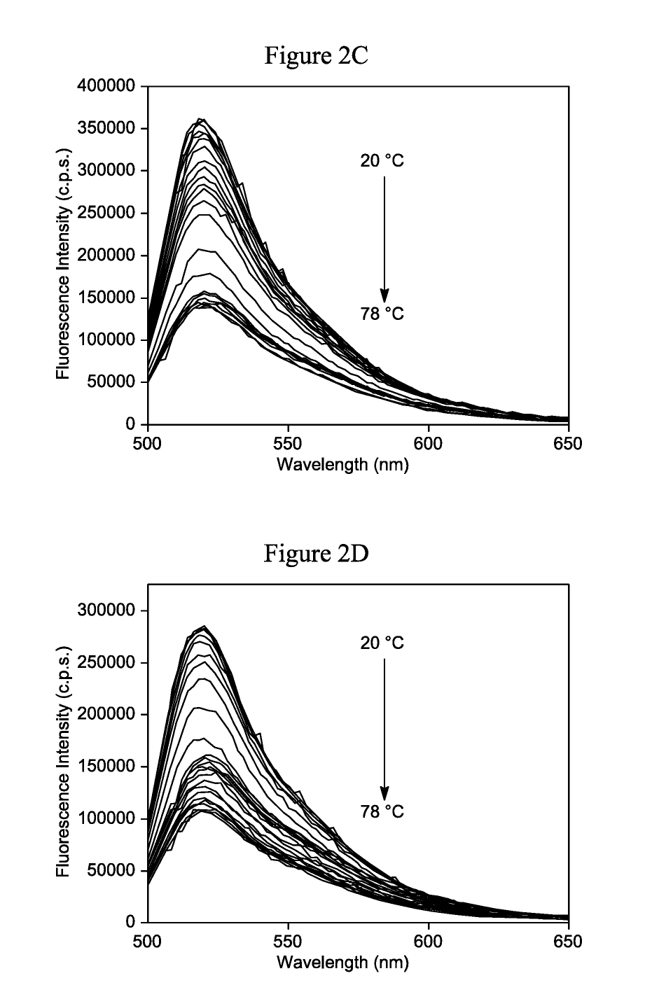

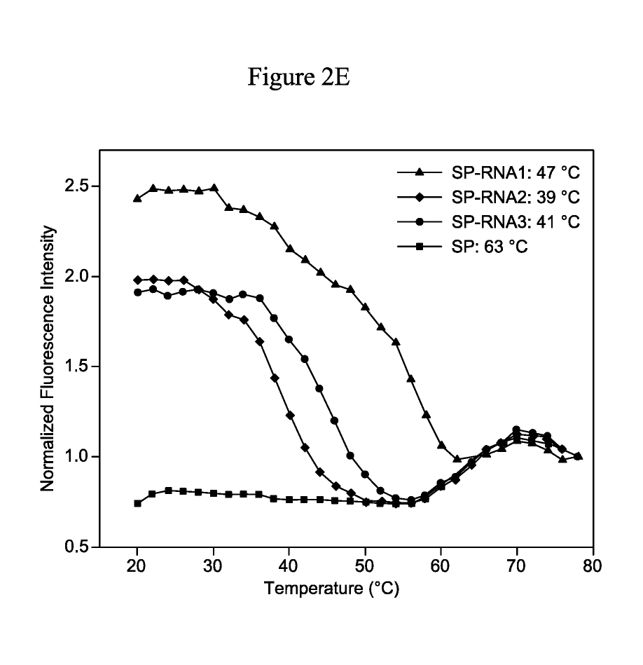

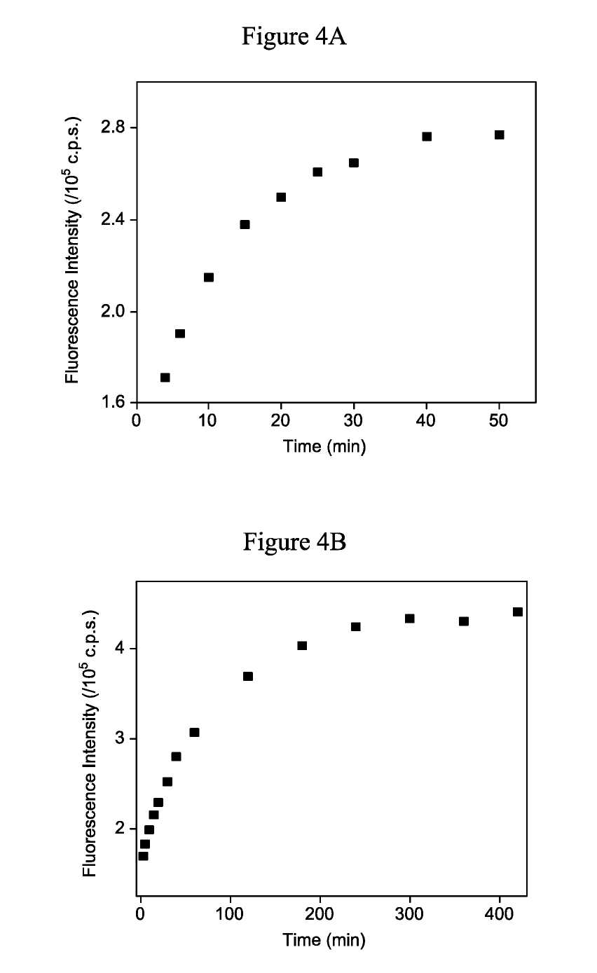

While the smart probe can hybridize to its target at ambient temperature in about 5 to 6 hours (FIG. 4B), the complete hybridization can be achieved in about 40-50 min at a temperature of 30-40° C., e.g., 37° C. (see FIG. 4B). The probe has high specificity for its target and hybridization with homologous target may be easily distinguished from hybridization to the intended target. FIG. 2E shows the melting profile of the smart probe with SEQ ID NO: 1, SEQ ID NO: 3 and SEQ ID NO: 4. SEQ ID NO: 3 and 4 differ from SEQ ID NO: 1 by one nucleotide. Clearly the duplex formed with SEQ ID NO: 1 produces significantly more intense fluorescence and has a significantly higher melting temperature than the duplexes of SEQ ID NO: 3 and 4. It should be noted, that the duplexes of the homologous target sequences have similar fluorescence intensity to that of hairpin structure of SEQ ID NO: 2 at 52° C., whereas the duplex of the true target has a significantly higher fluorescence intensity of about 45%-55% of the maximal fluorescence intensity at ambient temperature. Such differences may be utilized to distinguish between a duplex of true target and duplexes of other nucleic acids which are highly homologous in sequences to the true target.

In a preferred embodiment of the method, the target nucleic acid sequence is miRNA, preferably a miRNA marker for particular diseases such as but not limited to hereditary diseases, cancers, cardiovascular diseases, kidney diseases, alcoholism, obesity, viral infection and nervous system diseases. In a more preferred embodiment of the method, the miRNA is miR-21 which is upregulated in the blood of subjects having cancer and cardiovascular diseases. In the most preferred embodiment of the method the disease is a cancer selected from the group consisting of breast, ovary, cervix, colon, lung, liver, brain esophagus, prostate, pancreas, and thyroid.

As mentioned earlier, miR-21 of SEQ ID NO: 1 is a recognized biomarker for several human cancers. Thus, the method disclosed herein is a valuable method for detecting and confirming early diagnosis cancers, in particular, breast cancer, ovarian cancer, cervical cancer, colon cancer, lung cancer, liver cancer, brain cancer, esophageal cancer, prostate cancer, pancreatic cancer, and thyroid cancer. Also, the method can be utilized to screen for cancer in a suspected population prior to any diagnosis of cancer. For example, a subject with family history of breast or cervical cancer would greatly benefit from an early detection and diagnosis by monitoring the blood level of miR-21 of SEQ ID NO: 1 using the method disclosed herein. Also, screening patients for prostate and colon cancer using the method of the invention would be faster, easier, and more convenient than the rectal and endoscopic examination currently in use.

A third aspect of the invention is directed to a cancer diagnostic method comprising:
(a) contacting a biological sample of the subject with the smart probe disclosed herein,
(b) measuring the increase in the fluorescence of the probe,
(c) determining the concentration of miR-21 of SEQ ID NO: 1 in the biological sample, and
(d) diagnosing the presence of cancer when the concentration of miR-21 of SEQ ID NO: 1 in the subject is greater than the range of concentrations of miR-21 of SEQ ID NO: 1 determined in cancer free population

As used herein “biological sample” means any biological material including, but not limited to whole blood, blood serum, and cell culture. In a preferred embodiment, the biological sample is whole blood or blood serum, more preferably blood serum.

### EXAMPLE 1

### Materials:

The SP and all the single-stranded oligonucleotide target sequences are shown in Table I: the target sequence of miR-21 of SEQ ID NO: 1 (RNA1), and its analogous single-base mismatch sequences (RNA2 of SEQ ID NO: 3 and RNA3 of SEQ ID NO: 4), were custom-synthesized by Integrated DNA Technologies, BVBA, Belgium. The target oligonucleotides RNA1 of SEQ ID NO: 1, RNA2 of SEQ ID NO: 3, and RNA3 of SEQ ID NO: 4 were purified by standard desalting, while the SP of SEQ ID NO: 2 was purified by desalting and HPLC (Table I). All chemicals-magnesium chloride hexahydrate (MgCl₂.6H₂O), ethylenediaminetetraacetic acid (EDTA), and hydrochloric acid (BDH Chemicals Limited, UK); sodium hydroxide (Fluka AG, Switzerland); sodium chloride (Fisher Scientific Company, USA); Tris buffer (Sigma-Aldrich, USA) were used without any purification. All solutions were prepared with water obtained from a Barnstead Nanopure water purification system (Thermo Scientific, USA).

TABLE 1
List of oligonucleotide sequences

| | | SEQ |
|---|---|---|
| | | ID |
| Name | Sequence | NO: |
| miR-21 | 5′-rUrArGrCrUrUrArUrCrArGr | 1 |
| target | ArCrUrGrArUrGrUrUrGrA-3′ | |
| sequence | | |
| (RNA1)^{a} | | |
| Smart | 5′/6FAM/TCCCGAGGTCAACATCAG | 2 |
| probe | TCTGATAAGCTACCTCGGGAGGG-3′ | |
| (SP)^{b} | | |
| miR-21 | 5′-rUrArGrCrUrUrArUrCrArCr | 3 |
| mismatch | ArCrUrGrArUrGrUrUrGrA-3′ | |
| target | | |
| sequence | | |
| (RNA2)^{a,c} | | |
| miR-21 | 5′-rUrArGrCrUrUrArUrCrArGr | 4 |
| mismatch | ArCrUrGrArUrCrUrUrGrA-3′ | |
| target | | |
| sequence | | |
| (RNA3)^{a,c} | | |

^{a}The underlined bolded base in RNA2 and RNA3 is the single-base mismatch, whereby the corresponding rG in RNA1 has been replaced by rC.
^{b}The underlined bolded bases represent the stem strands, while the three Gs on the 3′ end are not part of the stem hybrid but are included as quenchers for the fluorophore
^{c}The difference between RNA2 and RNA3 is in the location of the single-base mismatch

Oligonucleotides of SEQ ID NO: 1 (RNA1), SEQ ID NO: 3 (RNA2), SEQ ID NO: 4 (RNA3) and SEQ ID NO: 2 (SP) were each dissolved in purified water and kept frozen at −20° C. until used. Upon thawing, they were diluted in 20 mM Tris, pH 7.5, buffer containing 2 mM EDTA, 200 mM NaCl and 3 mM MgCl₂ to give the desired oligonucleotide concentrations. The concentrations of the oligonucleotide solutions were confirmed by UV absorption measurements.

### EXAMPLE 2

### Absorbance and Fluorescence Measurements:

UV-Visible spectra of oligonucleotides were recorded with Genesys 10 S UV-Vis spectrophotometer, equipped with VISIONLite software (Thermo Scientific). All fluorescence measurements were carried out on a solution of 100 nM SP in the absence or presence of 300 nM target miRNA in Tris-EDTA buffer after the solution were incubated in the dark at room temperature for about 5 h. In concentration-dependent experiments, 500 nM SP in the presence of varying concentrations of RNA1 of SEQ ID NO: 1 target sequence ranging from 0.0 to 2000 nM were incubated in the dark at room temperature for about 5 h before fluorescence measurements. Prior incubation was not carried out for all kinetics experiments. All fluorescence spectra were measured using an FLS920 fluorescence spectrophotometer (Edinburgh Instruments, UK). The spectra were recorded in the range of 500-650 nm with an excitation wavelength of 490 nm and a single fluorescence measurement are observed at emission wavelength set at 520 nm. A 10 mm path length, submicro quartz cuvette (Cole-Parmer, USA) was used for the fluorescence measurements. Except for temperature-dependent experiments, all fluorescence spectra were recorded at room temperature in a 400 μL solution of the SP or SP-target hybridization mixture. Temperature-controlled fluorescence measurements were carried out on 400 μL solutions of SP of SEQ ID NO: 2 or SP-target duplexes, using a fluorescence spectrophotometer equipped with a TC125 temperature control unit (Quantum Northwest Inc., USA). The temperature varied in the range of 20-78° C. in 2° C. increments, with a settling time of 100 s. For kinetic experiments at ambient temperature, fluorescence spectra were measured at specified time intervals, immediately following the mixing of RNA1 of SEQ ID NO: 1 with SP of SEQ ID NO: 2 that had been thermostated for 5 min at ambient temperature. For kinetics experiments at 37° C., fluorescence spectra were measured at specified time intervals, immediately following the mixing of RNA1 of SEQ ID NO: 1 with SP of SEQ ID NO: 2 that had been thermostated for 5 min at 37° C. Also, the concentrations were maintained at 100 nM SP and 300 nM RNA1 for the kinetics experiments.

### EXAMPLE 3

### Structure of SP:

The 41 nucleotides SP of SEQ ID NO: 2, the target sequences miR-21 of SEQ ID NO: 1 (RNA1), and the variants thereof of SEQ ID NO: 3 and 4 containing a single base substitution of rG to rC (RNA2 and RNA3) (see Table I). The loop sequence of the SP of SEQ ID NO: 2 consists of 22 bases that are complementary to miR-21 of SEQ ID NO: 1. The stem hybrid consists of eight nucleotides on each side (FIG. 1). The 5′-end of the stem is labeled with a fluorescent dye, 6-FAM (6-carboxyflurescein). G-triplet, tandem of three guanine residues, at the 3′-end of the nucleic acid sequence are opposite to the fluorophore. The quenching is probably further enhanced by a second close by three guanine residues of the stem at the 3′-end which are separated from the terminal G-triplet by a single adenine residue. While the formation of G-tetraplexes is undesirable for our application, such structures may be harnessed to provide enhanced fluorescence quenching for sensitive analytical applications [Yang et al. Anal. Methods 2012. 4: 895-897, incorporated herein by reference].

SP of SEQ ID NO: 2 has been designed for maximum performance as a sensitive and specific probe for miR-21 of SEQ ID NO: 1. Based on the thermodynamic stability of MB (Bonnet et al. 1999), a functional MB or SP should have a stem duplex more thermally stable than the SP-target duplex. The stability is determined by the Tₘ of the stem duplex relative to that of SP-target duplex. The relative thermal stability requirement is met when the Tₘ of the stem hybrid is above ambient room temperature and at least 5° C. higher than that of the SP-target duplex. The thermal stability requirement of the smart probe ensures that the stem remains intact at room temperature and the fluorescence is significantly quenched. Also, it ensures that the SP can remain intact as a hairpin at relatively high temperatures when the SP-target duplex has melted. As shown in FIG. 1, the Tₘ of the SP of SEQ ID NO: 2 is 63° C. while that of the SP-RNA1 duplex is 47° C. Thus, the Tₘ of SP-RNA1 duplex is 16° C. lower than that of SP of SEQ ID NO: 2 alone showing the desired performance characteristics that confer exquisite specificity on the SP of SEQ ID NO: 2 have been achieved.

### EXAMPLE 4

### Thermal Transition Profile:

Smart probes should exhibit a conformational transition upon heating and the conformational change must be consistent with their design. When the temperature of the SP solution is increased, fluorescence should increase in a manner characteristic of the melting of double-stranded nucleic acids (Tyagi 1996, incorporated herein by reference). Thus, in order to ascertain the design and intended performance characteristics of the new SP, and to ensure its optimal performance, thermal transition profiles of the SP of SEQ ID NO: 2 alone and duplexes of SP-RNA1 (SEQ ID NO: 1), RNA2 (SEQ ID NO: 3) and RNA3 (SEQ ID NO: 4) were measured. In this respect, the fluorescence signal of the homogeneous solutions was measured while gradually increasing the temperature from 20° C. to 78° C. (FIG. 2E). FIG. 2E shows the thermal transition profiles of the SP of SEQ ID NO: 2 and SP of SEQ ID NO: 2 hybridized to the target complementary sequence miR-21 of SEQ ID NO: 1 (RNA1) and the single-base mismatch sequences of SEQ ID NO: 3 and 4 (RNA2 and RNA3, respectively).

At low temperatures, the SP of SEQ ID NO: 1 alone exists in the hairpin form and so the fluorescence of the fluorophore is sufficiently quenched by the proximal guanine residues at the 3′-end. Thus, there is minimal fluorescence due to photo-induced intramolecular electron transfer (energy transfer). On increasing the temperature to about 58° C., the stem hybrid begins to melt and the fluorophore begins to separate from the quenching guanine residues, and increase in fluorescence intensity is observed above 58° C. When the temperature is further increased, the fluorophore and the quencher moved further apart from each other, until the stem hybrid completely unwinds and the maximum fluorescence intensity is reached, explaining the increase in fluorescence intensity with increasing temperature between 58° C. and 70° C. Above 70° C., the SP of SEQ ID NO: 2 exists in a random coil conformation, which causes the fluorescence intensity to decrease somewhat. The overall thermal transition profile of the SP of SEQ ID NO: 2 alone is sigmoidal, which is consistent with the thermal transition behavior of a typical MB or SP (Tyagi et al. 1996, Bonnet et al. 1999, Oladepo et al. 2010, and Nair et al. 2013, both incorporated herein by reference). From the profile, the Tₘ of the SP of SEQ ID NO: 2 was determined to be 63° C. The Tₘ of SP of SEQ ID NO: 2 is dependent on the length of stem hybrid and the stem G-C content (Tyagi et al. 1996). The stem of the SP of SEQ ID NO: 2 consists of eight residues, five of which are G-C, excluding the quencher G-triplet-overhangs that is opposite to the fluorophore (see FIG. 1).

When the SP of SEQ ID NO: 2 is mixed with a threefold excess of miR-21 target RNA1 of SEQ ID NO: 1, a complementary sequence, an intense fluorescence is observed at low temperatures (circles, FIG. 2E). The target miR-21 of SEQ ID NO: 1 spontaneously hybridizes with the loop sequence of the SP of SEQ ID NO: 2, resulting in the spatial separation of the fluorophore and the quencher G-triplet. As the temperature gradually increases, the fluorescence intensity decreases indicating that the SP-RNA1 duplex is melting leading to the formation of the hairpin structure of the SP of SEQ ID NO: 1, which brings the fluorophore and guanine quenchers into close proximity. The observed fluorescence decrease continues with increasing the temperature until the target RNA1 of SEQ ID NO: 1 and SP of SEQ ID NO: 2 are separated, and formation of the hairpin structure placing the fluorophore in close proximity to the G-triplet and displaying lowest fluorescent intensity in the profile (circles, FIG. 2E). When the temperature is further increased, the stem of the hairpin begins gradually melting with concomitant increase in the fluorescence intensity. The melting of the hairpin stem produces a random coil leading to a limited fluorescence signal. The Tₘ of SP-RNA1 was determined to be 47° C., giving a Tₘ difference (ΔTₘ) of 16° C. with respect to SP of SEQ ID NO: 2 alone. The ΔTₘ value suggests excellent recognition of the target RNA1 of SEQ ID NO: 1 by the SP of SEQ ID NO: 2. When the fluorescence intensity of the SP-RNA1 duplex is compared with that of SP of SEQ ID NO: 2 alone, there is about a threefold increase in fluorescence intensity at 20° C. This intensity increase is comparable to what was previously reported for smart probes (Stohr et al. 2005, and Oladepo et al. 2010). Thus, the SP of SEQ ID NO: 2 disclosed herein displays excellent target recognition with high specificity.

In the presence of a threefold excess of a single-base mismatch sequence RNA2 of SEQ ID NO: 3 over SP of SEQ ID NO: 2, a thermal transition profile similar to that of SP-RNA1 duplex was observed (diamonds, FIG. 2E). However, the maximum fluorescence intensity of the hybrid is lower than that of SP-RNA1 at low temperature indicating lower stability of the SP-RNA2 duplex, which contains at least one base mismatch. Such a mismatch target sequence should melt at temperatures below that of the complementary target RNA1 of SEQ ID NO: 1, at which temperature the SP of SEQ ID NO: 2 will remain a hairpin and therefore, produces significantly quenched fluorescence. In fact, this was the case as the observed Tₘ of SP-RNA2 duplex was 39° C., which is 8° C. lower than that of SP-RNA1 duplex (diamonds, FIG. 2E). Similarly, when mixing SP of SEQ ID NO: 2 with a threefold excess of another single-base mismatch RNA3 of SEQ ID NO: 4, a low intensity signal was observed with a thermal profile similar to that of SP-RNA2 (hexagons, FIG. 2E) and a Tₘ of 41° C. The results show high selectivity and specificity of the new SP of SEQ ID NO: 2 for detecting the target miR-21 of SEQ ID NO: 1 and demonstrate its ability to distinguish between analogous sequences that differ by only one nucleotide. Also, they show that the SP of SEQ ID NO: 2 is capable of discriminating against any mismatch sequence irrespective of the location of the mismatch. Given that miRNAs have similar sequences; this new SP of SEQ ID NO: 2 has the potential to discriminate the target miR-21 of SEQ ID NO: 1 from other miRNAs with as little as a single base mismatch.

### EXAMPLE 5

Enhanced Temperature—Dependent Discrimination:

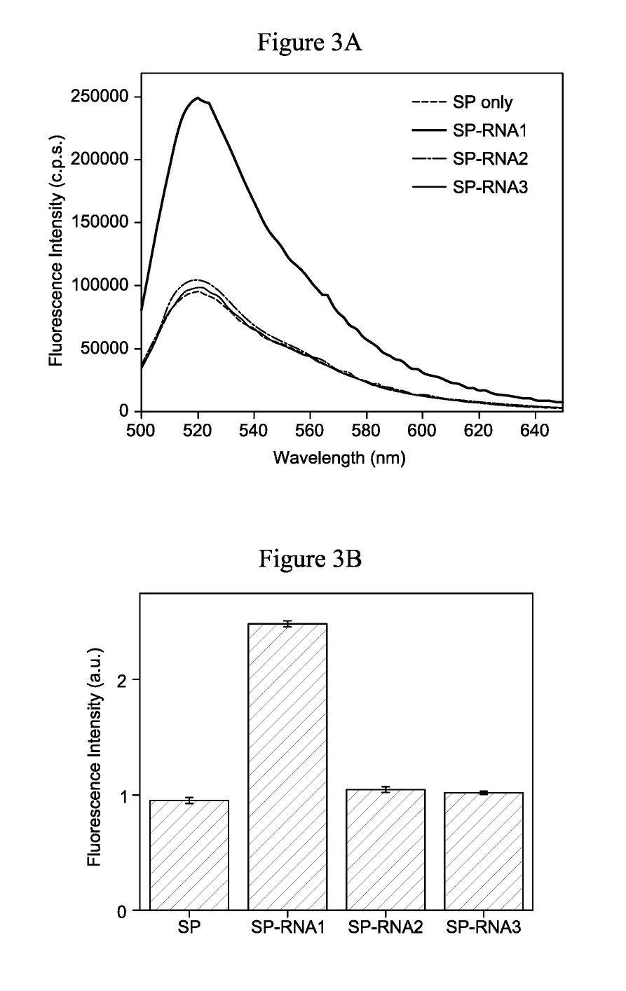

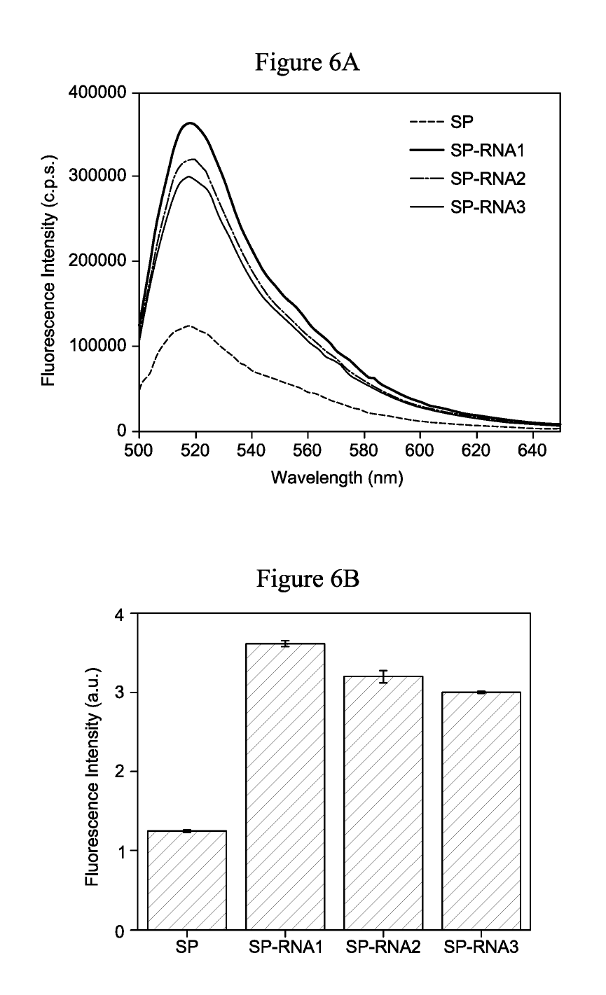

A closer look at FIG. 2E suggests several possible methods of discriminating between the target miR-21 of SEQ ID NO: 1 (RNA1), and the single-base mismatch sequences of RNA2 of SEQ ID NO: 3 and RNA3 of SEQ ID NO: 4. The methods include, but are not limited to: measurement of the thermal transition profiles to determine the Tₘ; single-point measurement of fluorescence intensities at ambient temperature; and single-point measurement of fluorescence intensities at a high temperature of about 52° C. The most preferred method to discriminate between RNA1 of SEQ ID NO: 1 duplex with SP of SEQ ID NO: 2 and those of the two mismatch duplexes RNA2 of SEQ ID NO: 3 and RNA3 of SEQ ID NO: 4 is by measuring the fluorescence intensities of duplexes around 52° C. At 52° C., the fluorescence intensity produced by SP-RNA2 and SP-RNA3 duplexes are significantly quenched similar to that of SP of SEQ ID NO: 2 alone (see FIG. 2E). In contrast, SP-RNA1 duplex gives a much higher intensity fluorescence signal at same temperature. This observation is consistent with the relative Tₘ's of the three duplexes. FIG. 2E shows that, at temperatures significantly higher than the Tₘ of SP-RNA2 and SP-RNA3 duplex (about 40° C.), the duplexes are melted and denatured leading to the formation of the hairpin structure of SP of SEQ ID NO: 2, and hairpin structure would remain as long as the temperature is lower than the Tₘ of the SP of SEQ ID NO: 2 alone (FIG. 2E). As long as the temperature is within about ±5° C. of the Tₘ of SP-RNA1 duplex such that this duplex is still mostly intact, there will be significant fluorescence (FIGS. 3A & B). Thus, when the fluorescence signal was measured at 52° C. for all three duplexes (SP-RNA1, SP-RNA2, and SP-RNA3), the signal produced by SP-RNA1 duplex is about 250% of those of SP-RNA2 and SP-RNA3 (FIG. 3). At 52° C., 50% of the SP of SEQ ID NO: 2 is hybridized with RNA1 of SEQ ID NO: 1 and consequently, an intense fluorescence is observed. In contrast, very little if any of the SP of SEQ ID NO: 2 is hybridized with RNA2 of SEQ ID NO: 3 and RNA3 of SEQ ID NO: 4 and the fluorescence is significantly quenched. The method being presented here is for an in vitro detection of miR-21 of SEQ ID NO: 1 by the SP of SEQ ID NO: 2 and the single fluorescence measurement at 52° C. would make it a method of choice for a clinical diagnostics. Thus, the probe of the disclosure has been successfully used in a simple homogeneous assay, and it therefore constitutes a simple, mix-and-read assay for fast detection of miR-21 of SEQ ID NO: 1. Similar measurements were made at 20° C., and the signal generated by SP-RNA2 duplex is about 90% that of SP-RNA1 duplex, while that generated by SP-RNA3 is about 85% that of SP-RNA1 (FIGS. 6A and 6B). Thus, the discrimination at this low temperature is only marginal compared to 52° C.

### EXAMPLE 6

### Hybridization Kinetics:

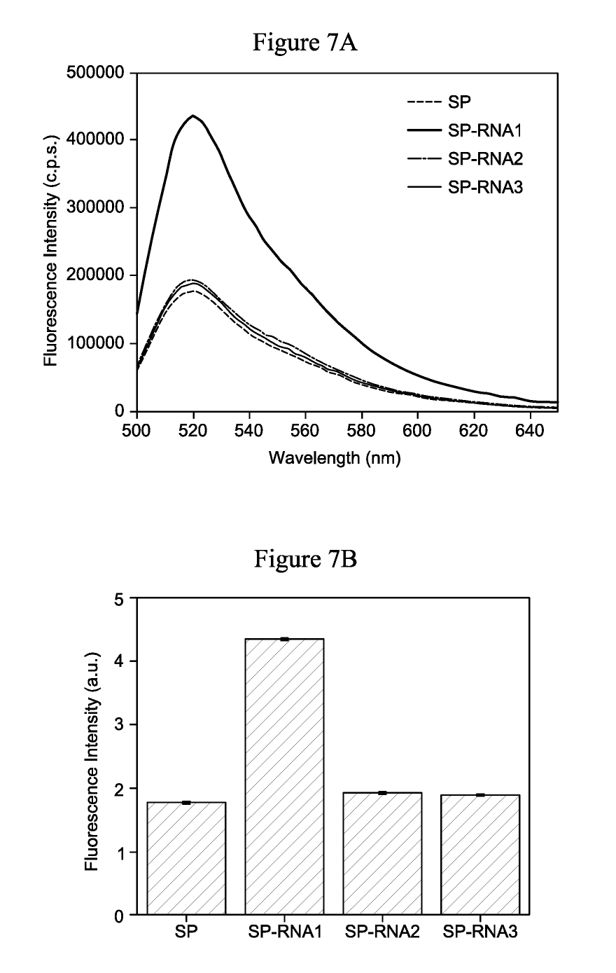

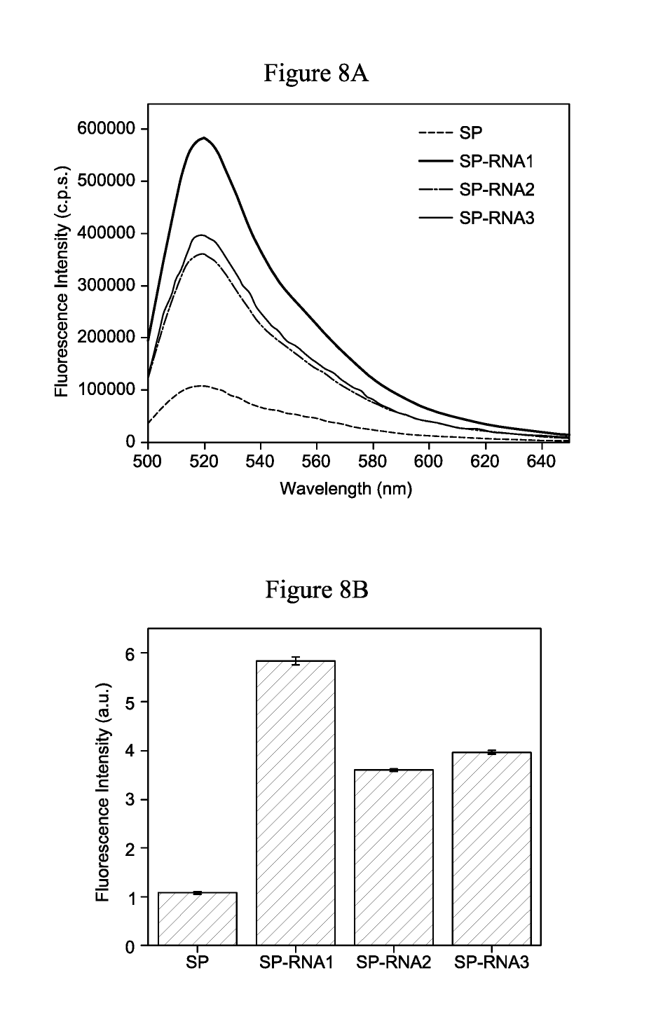

At ambient temperature, hybridization of the SP of SEQ ID NO: 2 with target miR-21 of SEQ ID NO: 1 (RNA1) takes about 5 h to complete (FIG. 4B), which is consistent with similar probe-target hybridizations involving MBs and SPs. [El-Yazbi et al. 2013). However, 5 h is relatively a long time, if this assay method is adapted for rapid diagnostic applications. To accelerate the hybridization, time-dependent experiments were carried out at 37° C. As shown in FIG. 4A, complete hybridization at 37° C. is achieved in only 40 min, which is about seven times faster than that at ambient temperature. Thus, the homogeneous assay can in fact be completed in about 40 min if the SP of SEQ ID NO: 2 is hybridized with miR-21 of SEQ ID NO: 1 at 37° C. Also, single-point measurements of fluorescence intensities were carried out, following hybridization at 37° C. Single-point fluorescence intensities measured at 52° C. following incubation at 37° C. gave a signal of 230% for SP-RNA1 duplex compared to those of SP-RNA2 and SP-RNA3 duplexes (FIGS. 7A and 7B). The level of discrimination is consistent with that obtained following hybridization at ambient temperature (FIG. 3B), but the analysis can be completed much faster at this higher incubation temperature. Similar fluorescence signal measurements at 37° C. following incubation at same temperature only provides a signal of about 150-160% for SP-RNA1 compared to SP-RNA2 and SP-RNA3 (FIGS. 8A and 8B). So, for a rapid detection, the new SP of SEQ ID NO: 2 can be hybridized with miR-21 of SEQ ID NO: 1 at 37° C., followed by a single-point fluorescence measurement at 52° C. The hybridization at 37° C. provides faster measurement, high specificity, and sufficient discrimination between the target miR-21 of SEQ ID NO: 1 and single-base mismatched sequences.

### EXAMPLE 7

### Concentration-dependent Measurements:

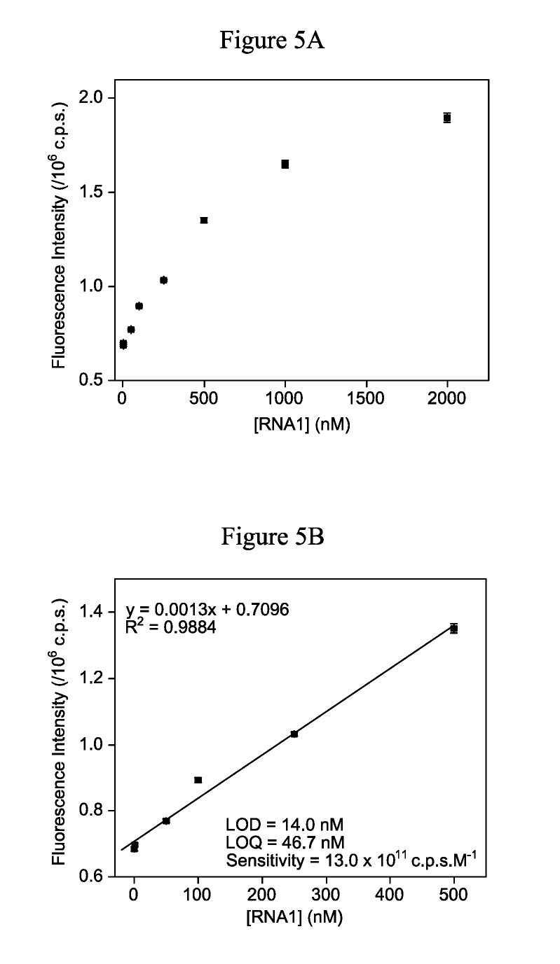

In order to determine the sensitivity of the SP of SEQ ID NO: 2, concentration-dependent experiments were carried out. Varying concentrations of RNA1 of SEQ ID NO: 1 was mixed with 500 nM SP, followed by fluorescence signal measurements. The fluorescence signal linearly increased with increasing the concentration of RNA1 of SEQ ID NO: 1 until RNA1 of SEQ ID NO: 1 concentration reached 500 nM, i.e., 1:1 SP/RNA1 molar equivalent. Thus, the calibration graph is linear in the concentration range of 0-500 nM RNA1 of SEQ ID NO: 1 (see FIG. 5B), beyond which point higher concentrations of RNA1 of SEQ ID NO: 1 produced little change in fluorescence intensity (FIGS. 5A and 5B). The limit of detection (LOD) and limit of quantitation (LOQ) were found to be 14.0 nM and 46.7 nM, respectively (FIG. 5B). The LOD and LOQ were determined to be 3 sbl/m and 10 sbl/m, respectively, where sbl is the standard deviation of ten replicate measurements of the blank (500 nM SP) and m is the slope of the calibration curve (FIG. 5B). The sensitivity represents the slope of the calibration curve and was determined to be 13.0×10¹¹ c.p.s. M⁻¹. These values are consistent with previously reported values for MB and SP (El-Yazbi et al. 2013, Stohr et al. 2005, and Oladepo et al. 2010).

### EXAMPLE 8

Comparison with Other Methods:

The mix-and-read homogeneous assay method using the SP probe of SEQ ID NO: 1 disclosed herein is simple, fast, and specific, with sensitivity in the nanomolar concentration range. This method is similar to PCR-based methods in terms of selectivity and specificity. However, given the time consuming and labor intensive nature of PCR-based methods, the SP-based method is the method of choice due to its simplicity and rapidity. The SP of SEQ ID NO: 2 method described herein has several advantages compared to microarray method and northern blotting method, as these methods suffer from low sensitivity and are cumbersome (Cissel et al. 2008, Catuogno et al. 2011, Liu et al. 2012, and Wang et al. 2012). Similarly, electrochemical and colorimetric detections methods have lower detection limits (Kilic et al. 2012, and Park et al. 2014) than the homogeneous assay method of the invention which is less complicated and faster to carry out. The detection method using the SP of SEQ ID NO: 2 has nanomolar sensitivity, which is similar to that of SERS-based method (Guven et al. 2014), yet it is simpler and less cumbersome. While bioluminescence-based method provides a lower LOD than that of the SP-based method, the latter method is less involved and faster to carry out. The SP-based method of the invention may not rival the detection sensitivity of nucleic acids amplification methods, which ranges from picomolar to attomolar levels (Liu et al. 2012, Wang et al. 2012, Duan et al. 2013, and Cheng et al. (2016). These amplification methods have multiple complicated steps, which may make them less attractive compared to the SP-based method of the invention. It should be mentioned that several amplification methods are also based on hairpin probes that are similar in structure to SP and MB (Zhang et al. 2016, Wang et al. 2012, and Duan 2013), and thus, the extra sensitivity provided by such amplification methods comes from the amplification steps. Overall, the new SP-based homogeneous assay is simple, less cumbersome, and fast, and provides high specificity for the target miR-21 of SEQ ID NO: 1 with a reasonably good sensitivity. It should be noted that the detection sensitivity reported here may be further improved by adding a nucleic acid amplification step to the assay method described herein ((Liu et al. 2012, Zhang et al. 2016, Wang et al. 2012, Zhou et al. 2014, and Duan 2013). Thus, the simple, mix-and-read homogeneous assay being described herein is a valuable diagnostic tool.

A sensitive and specific smart probe for the detection and quantification of a target nucleic acid is disclosed. The probe is particularly useful for detecting and quantifying miRNA, in particular, miR-21 of SEQ ID NO: 1, a recognized biomarker for several cancers. The thermal transition profiles of the smart probe and its target duplexes suggest that it has good performance characteristics and possesses excellent sequence specificity. The smart probe recognizes its target with high specificity and the target sequence can be distinguished from other nucleic acids with highly homologous sequences to that of the target sequence. With sufficient refinement the method can be adapted for in vivo detection of the target miRNA. The assay reported herein gives a LOD of 14.0 nM, a LOQ of 46.7 nM, and a sensitivity of 13.0×10¹¹ c.p.s. M⁻¹. While these values are consistent with literature values for MB- and SP-based. assays, they are fairly modest compared to other methods. However, the SP-based homogeneous assay presented here is simpler and faster, and it can be combined with amplification methods such as quadratic isothermal amplification to improve the sensitivity. The disclosed method for detecting a target nucleic acid is efficient and can be completed in about 40 minutes for detecting miR-21 of SEQ ID NO: 1 at 37° C.

1: A nucleic acid smart probe comprising:
(a) a complementary nucleic acid sequence to a target sequence of interest,
(b) a nucleic acid sequence A of 5 to 15 nucleotides bonded to the 3′-end of the complementary sequence of (a),
(c) a nucleic acid sequence B of 5 to 15 nucleotides bonded at the 5′-end of the complementary sequence of (a),
(d) a GGG sequence bonded at the 5′- or the 3′-end of the nucleic acid sequence A or B, and
(e) a fluorescence moiety bonded at the 5′- or the 3′-end of the nucleic acid sequence A or B,
wherein the nucleic acid sequences A and B are complementary sequences and form a stem of a hairpin structure.

2: The nucleic acid probe of claim 1, wherein the fluorescence moiety is at the 5′-end and the GGG sequence at the 3′-end.

3: The nucleic acid probe of claim 1, wherein the fluorescence moiety is at the 3′-end and the GGG sequence at the 5′-end.

4: The nucleic acid probe of claim 1, wherein the melting temperature Tₘ of the probe is in the range 57° C. to 67° C.

5: The nucleic acid probe of claim 1, wherein the melting temperature Tₘ of the probe-target nucleic acid is in the range 43° C. to 50° C.

6: The nucleic acid probe of claim 1, wherein the target nucleic acid of interest is SEQ ID NO: 1.

7: The nucleic acid probe of claim 1, wherein the nucleic acid of (c) is SEQ ID NO: 2.

8: The nucleic acid probe of claim 1, wherein the fluorescence moiety is 6-carboxyflurescein.

9: The nucleic acid probe of claim 1, wherein the nucleic acid sequence is SEQ ID NO: 2, the fluorescence moiety is 6-carboxyflurescein at the 5′-end, and the GGG sequence is at the 3′-end.

10: A method of detecting and/or quantifying a target nucleic acid sequence in a biological sample comprising:
(a) contacting the probe of claim 1 with a biological sample, and
(b) measuring the increase in fluorescence intensity.

11: The method of claim 10, wherein the target nucleic acid is miRNA.

12: The method of claim 11, wherein the miRNA is miR-21 of SEQ ID NO: 1.

13: The method of claim 12, wherein the smart probe has a nucleic acid sequence having at least 60% sequence identity to SEQ ID NO: 2.

14: The method of claim 12, wherein measuring the fluorescence at a temperature between the Tₘ of the probe and Tₘ of the probe-target nucleic acid duplex.

15: The method of claim 12, wherein measuring the fluorescence at a temperature in the range 45° C. and 56° C.

16: A method of diagnosing or detecting cancer in a subject comprising:
(a) contacting a biological sample of the subject with the probe of claim 9,
(b) measuring the increase in the fluorescence of the probe,
(c) determining the concentration of miR-21 of SEQ ID NO: 1 in the biological sample, and
(d) diagnosing the presence of cancer when the concentration of miR-21 of SEQ ID NO: 1 in the subject is greater than the range of concentrations of miR-21 of SEQ ID NO: 1 determined in cancer free population.

17: The method of claim 16, wherein the cancer is selected from the group consisting of leukemia, breast, colon, pancreatic, lung, prostate, liver, and stomach cancers.

18: The method of claim 13, wherein measuring the fluorescence at a temperature in the range 45° C. and 56° C.

19: The method of claim 13, wherein the subject is a mammal.

20: the method of claim 19, wherein the mammal is human.


SEQUENCE LISTING (4 sequences)

SEQ ID NO: 1 - RNA, 22 nt, Homo sapiens
uagcuuaucagacugauguuga

SEQ ID NO: 2 - DNA, 41 bp, Artificial Sequence
  Description of Artificial Sequence Synthetic probe
tcccgaggtcaacatcagtctgataagctacctcgggaggg

SEQ ID NO: 3 - RNA, 22 nt, Artificial Sequence
  Description of Artificial Sequence Synthetic oligonucleotide
uagcuuaucacacugauguuga

SEQ ID NO: 4 - RNA, 22 nt, Artificial Sequence
  Description of Artificial Sequence Synthetic oligonucleotide
uagcuuaucagacugaucuuga